(19) United States
(12) Patent Application Publication
(10) Pub. No.: US 2010/0210008 A1
(43) Pub. Date: Aug. 19, 2010

(54) Configurable Microfluidic Substrate Assembly

(75) Inventors: David Strand, Sherborn, MA (US); David Barrow, Cardiff (GB)

Correspondence Address: BANNER & WITCOFF, LTD., 28 STATE STREET, SUITE 1800, BOSTON, MA, 02109-1701, US

(73) Assignee: Protasis Corporation, Marlborough, MA (US)

(21) Appl. No.: 12/618,349

(22) Filed: Nov. 13, 2009

Related U.S. Application Data
(63) Continuation of application No. 10/537,690, filed on Aug. 3, 2006, now abandoned.
(60) Provisional application No. 60/431,039, filed on Dec. 5, 2002.

(51) Int. Cl. C12M 1/34; H01J 49/26; G01R 33/20; G01F 1/00; G01L 7/00; G01N 29/02; G01K 13/00; G01R 27/08; G01R 19/00; G01T 1/10; G01N 33/00; G01N 27/26

(52) U.S. Cl. 435/287.1; 250/281; 324/307; 73/861; 73/700; 73/64.53; 73/24.01; 374/141; 324/722; 324/76.11; 250/361 C; 422/68.1; 204/400; 204/450; 374/E13.001

(57) ABSTRACT

A microfluidic substrate assembly includes a substrate body having at least one fluid inlet port. At least one microscale fluid flow channel in the substrate is in fluid communication with the inlet port for transport of a fluid to be tested. The substrate body also has a plurality of sockets, with each of one or sockets configured to receive an operative component. At least one socket is in communication with the microscale fluid flow channel.

### FIELD OF THE INVENTION

The present invention relates to fluid-handling substrate devices and, more particularly, to microfluidic substrate assemblies and to methods for making certain preferred embodiments of microfluidic substrate assemblies.

### BACKGROUND

Systems for biochemical, chemical, and molecular analysis can be miniaturized as substrates with multifunctional capabilities including, for example, chemical, optical, fluidic, electronic, acoustic, and/or mechanical functionality. Miniaturization of these systems offers several advantages, including increased portability and lower production cost. Such devices can be fabricated from a diverse ensemble of materials including, for example, plastics or polymers, metals, silicon, ceramics, paper, and composites of these and other materials. Typically, such substrates include fluid channels extending within them for the transport and/or analysis of fluids or components contained in the fluids. Additionally, the channels may contain fragile or environmentally sensitive structures, such as materials, architecture and/or devices used for analyzing the fluids or components contained therein.

Currently known miniaturized fluid-handling devices have not met all of the needs of industry. Traditionally, a miniaturized fluid handling substrate is designed and produced for a specific application. This may require specific machining, casting, and welding, as well as incorporation of specific devices within the substrate body. A change in the specific application typically requires significant retooling and consequent additional costs. Additionally, if a technician in the field realizes the need for additional functionality or essential features after production, the already produced devices are no longer useful and must be replaced. So, not only are there costs incurred with redesign, retooling, and additional manufacturing, but there can also be a loss of time and money already spent on the previously, produced substrates.

Therefore, there exists a need in the art for improved fluid-handling substrates, and for cost effective methods of increasing functionality of the substrates. It is a general object of the present invention to provide improved fluid handling substrates, particularly microfluidic substrates, and methods for increasing their functionality. These and other objects of the invention will be more fully understood from the following disclosure and detailed description of certain preferred embodiments of the invention.

### SUMMARY OF THE INVENTION

In accordance with a first aspect, a microfluidic substrate assembly includes a substrate body having at least one fluid inlet port. At least one microscale fluid flow channel within the substrate is in fluid communication with the inlet port for transport of a fluid to be tested. The substrate body also has a plurality of component sockets, with each such component socket configured to receive an operative component. At least one socket is in communication with the microscale fluid flow channel. Optionally, the substrate body also has one or more additional sockets not configured to receive an operative component.

In certain preferred embodiments, at least some of the sockets configured to receive operative components are in fluid and/or electrical (e.g., electrical power, data, etc.) communications with each other via suitable flow channels or electrically conductive pathways in the substrate. For good design flexibility, as well be further understood from the detailed description below, it is generally preferred that all of the sockets adapted to receive an operative component are in fluid communication and electrical communication with each other.

As described further below, the operative components employed in any particular embodiment of the microfluidic substrate assemblies disclosed here typically are, selected to perform, cooperatively with each other, the process for which the microfluidic substrate device is intended. An individual operative component, preferably, performs a single standardized function on a fluid being processed by the substrate assembly, such as filtering, analyte focusing or concentrating, sensing, testing (e.g., pH, optical properties, conductivity, etc.). A desired change in the process can be implemented by adding, deleting or substituting operative components in the substrate sockets. For certain intended uses or applications, fewer than all available sockets will be used. Unused sockets can be plugged or left empty, depending on the design of the substrate and of the operative components employed.

In certain preferred embodiments of the microfluidic substrate assemblies disclosed here, at least some of the substrate sockets have the same configuration, that is, they share a single, common configuration, such that any correspondingly configured operative component, i.e., any component having a form adapted to be received by such socket configuration can be used in any of those sockets. Preferably the socket configuration is a standard configuration, that is, the same socket configuration is used for multiple sockets in multiple microfluidic substrate assemblies.

Certain preferred embodiments further comprise multiple operative components, each operative to perform a different function, and each having the same interface configuration adapted to be operatively received by the aforesaid standard socket configuration. As noted above, the selection of particular operative components and their position in the microfluidic substrate assembly will be largely determined by the particular application for which the assembly is intended.

In accordance with another aspect, a microfluidic substrate assembly includes a generally planar multi-layer laminated substrate having at least one fluid inlet port and at least one microscale fluid flow channel at each of more than one level within the multi-layer substrate, in fluid communication with the inlet port for transport of a fluid to be tested. At least one microscale fluid flow channel “via” extends between levels within the multi-layer laminated substrate for fluid communication between microscale fluid flow channels on different levels. The substrate body also has a plurality of sockets, with each socket configured to receive an operative component. At least one such socket is in fluid and/or electrical communication with at least one of the microscale fluid flow channels.

In accordance with a further aspect, a microfluidic substrate assembly includes a substrate body having at least one fluid inlet port and at least one microscale fluid flow channel within the substrate body in fluid communication with at least one fluid inlet port for transport of a fluid to be tested. The substrate body has a plurality of sockets, with each configured to receive an operative component and in communication with at least another socket. At least one socket is in communication with the microscale fluid flow channel. At least one operative component is mounted in a corresponding one of the plurality of sockets.

### BRIEF DESCRIPTION OF THE FIGURES

Certain preferred embodiments are described below with reference to the attached drawings in which:

FIG. 1 is a schematic plan view, shown partially in section, of one embodiment of a microfluidic substrate assembly in accordance with the present invention.

FIG. 2 is a schematic exploded perspective view of a housing encapsulating a microfluidic substrate assembly in accordance with another preferred embodiment, shown with an operative component and a plug for mounting in corresponding sockets of the microfluidic substrate assembly.

FIG. 3 is a schematic plan view of a preferred embodiment of the microfluidic substrate assembly of FIG. 2, shown with operative components mounted to the microfluidic substrate assembly.

FIG. 4 is a schematic section view showing microscale fluid flow channels and data channels at multiple levels within the microfluidic substrate assembly of FIG. 2.

FIGS. 5A-D are schematic section views of alternative embodiments of microfluidic substrate assemblies disclosed here, showing various configurations for microscale fluid flow channels and data channels within the microfluidic substrate assembly.

FIG. 6 is a schematic exploded perspective view of an alternative embodiment of the substrate of the microfluidic substrate assembly of FIG. 1, shown secured between two rigid plates.

FIGS. 7A-B are schematic section views showing assembly of a substrate of the microfluidic substrate assembly of FIG. 1 in accordance with a preferred embodiment.

FIGS. 8A-C are schematic section views showing assembly of a substrate of the microfluidic substrate assembly of FIG. 1 in accordance with another preferred embodiment.

FIGS. 9A-C are schematic section views showing assembly of a substrate of the microfluidic substrate assembly of FIG. 1 in accordance with yet another preferred embodiment.

FIGS. 10A-B are schematic diagrams of a high-pressure liquid chromatography system incorporating the microfluidic substrate assembly of FIG. 1 in accordance with yet another preferred embodiment.

FIG. 11 is a schematic perspective view of an analytical system using a microfluidic substrate assembly in accordance with a preferred embodiment.

FIG. 12 is a schematic perspective view of a multi-layer laminated manifold in fluid communication with a microfluidic substrate assembly, in accordance with a preferred embodiment.

FIG. 13 is a schematic perspective view of a multi-layer laminated manifold in fluid communication with a multi-layer laminated substrate assembly and with a device for generating fluid flow, in accordance with a preferred embodiment.

FIG. 14 is a schematic perspective view of a second embodiment of an analytical system in communication with a microfluidic substrate assembly.

It will be recognized by those skilled in the art that the microfluidic substrate assemblies shown in the figures are not necessarily to scale. Additionally, references to orientation, e.g. top, bottom and the like, are for convenience purposes only and are not intended to limit the disclosure in any manner. One skilled in the art, given the benefit of this disclosure, will be able to select and design microfluidic substrate assemblies having dimensions and geometries suitable for a desired use and suitable for use in any orientation.

### DETAILED DESCRIPTION OF CERTAIN PREFERRED EMBODIMENTS

Numerous embodiments of the present invention are possible and will be apparent to those skilled in the art, given the benefit of this disclosure. The detailed description provided herein, for convenience, will focus on certain illustrative and exemplary embodiments.

Preferred embodiments of the devices disclosed herein can be utilized, for example, in any of a wide range of automated tests for the analysis and/or testing of a fluid. As used here, the term “fluid” refers to gases, liquids, supercritical fluids and the like, optionally containing dissolved species, solvated species and/or particulate matter. Testing or analysis of a fluid, as used herein, has a broad meaning, including any detection, measurement or other determination of the presence of a fluid or of a characteristic or property of the fluid or of a component of the fluid, such as particles, dissolved salts or other solutes or other species in the fluid. Especially preferred embodiments of fluid handling devices disclosed here are operative to perform liquid separation analyses. That is, the devices perform or are adapted to function in a larger system which performs any of various different fluid separation test or analysis methods, typically along with ancillary and supporting operations.

In preferred embodiments, substrate assemblies disclosed here are “microfluidic” in that they operate effectively on microscale fluid samples, typically having fluid flow rates as low as about 1 ml/min., preferably about 100 ul/min. or less, more preferably about 10 ul/min. or less, most preferably about 1 ul/min. or less, for example, about 100 nanoliters/min. Total fluid volume for a liquid chromatography (LC) or other such fluid separation method performed using substrate assemblies disclosed here, e.g., in support of a water quality test to determine the concentration of analytes in the water being tested, in accordance with certain preferred embodiments, can be as small as about 10 ml or less, or 1 ml or less, preferably 100 microliters, more preferably 10 microliters or even 1 microliter or less, for example, about 100 nanoliters. As used herein, the term “microfluidic” also refers to flow passages or channels and other structural elements of the substrate body. For example, the one or more microscale fluid flow channels, or micro channels, of the substrate preferably have a cross-sectional dimension (diameter, width or height) preferably between about 500 microns and about 100 nanometers. Thus, at the small end of that range, the microchannel has a cross-sectional area of about 0.01 square microns. The microfluidic nature of the substrate assemblies disclosed here provides significant commercial advantage. Less sample fluid is required, which in certain applications can present significant cost reductions, both in reducing product usage (for example, if the test sample is taken from a product stream) and in reducing the waste stream disposal volume. Samples can be concentrated either by operative components of the microfluidic substrate assembly or prior to separation and/or entry into the microfluidic substrate assembly. In addition, the microfluidic substrate assemblies can, in accordance with preferred embodiments, be produced employing micro electromechanical systems (MEMS) and other known techniques suitable for cost effective manufacture of miniature high precision devices. It should be recognized that the substrate body may, in certain embodiments, have operative features, such as fluid channels, reaction chambers or zones, accumulation sites, etc. that are larger than microscale.

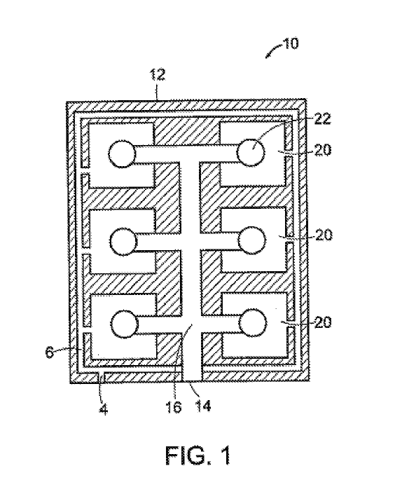

In accordance with certain preferred embodiments, as seen in FIG. 1, a microfluidic substrate assembly 10 (also referred to herein as a “cartridge”) includes a substrate body 12, defining a fluid inlet port 14, a microscale fluid flow channel 16, and a plurality of sockets 20. Microscale fluid flow channel 16 is in fluid communication with inlet port 14. Sockets 20 are in fluid communication via ports 22 with microscale fluid flow channel 16.

Substrate body 12 can be manufactured from numerous materials. Preferably the material should be capable of withstanding high pressure and harsh environments. Examples of suitable materials include plastics, polymers, metals, silicon, ceramics, etc. In a preferred embodiment, the substrate is formed of polyetheretherketone (PEEK). In certain preferred embodiments, the substrate body 12 is a multi-layer laminate. The layers of the substrate can be made from any of the above materials or combination thereof. In another embodiment, substrate body 12 further defines a fluid reservoir in fluid communication with the microscale fluid flow channel 16.

Inlet port 14 receives a fluid to be tested from an external source. There may be more than one inlet port 14 for receiving multiple fluids to be tested, and/or buffers, solvents, purging or flushing fluids, etc. Inlet port 14 optionally includes filtering for the fluid to be received. The microscale fluid flow channel 16 (also referred to in some cases as a microfluidic channel or a microchannel) is in fluid communication with the inlet port 14. The microscale fluid flow channel 16 receives the fluid from inlet port 14 and transports it for testing. In certain embodiments, there may be multiple microscale fluid flow channels for transporting separate fluids. The multiple channels may or may not be interconnected.

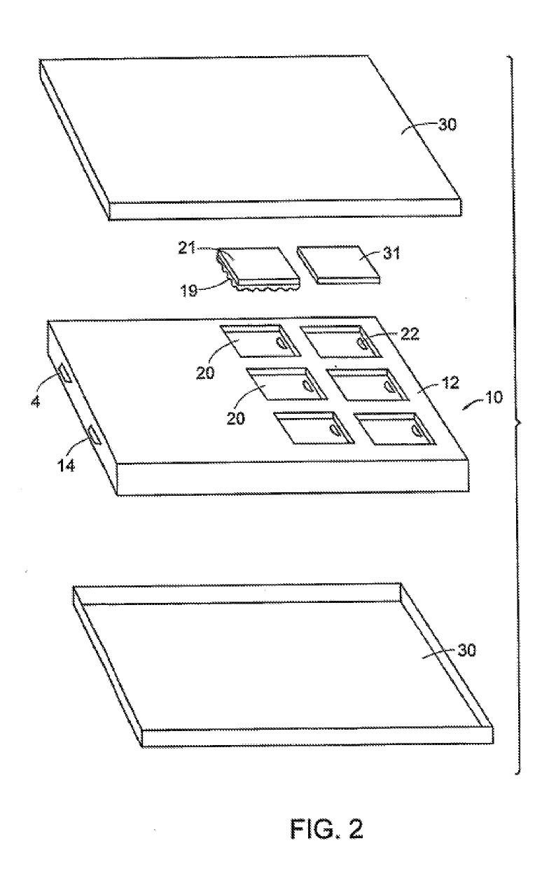

Sockets 20 each is designed for receiving an operative component 21 (shown and described in greater detail below with respect to FIG. 2), which provides additional functionality. One or more of the sockets 20 are configured to provide communication between an operative component 21 and the microscale fluid flow channel 16. In accordance with certain preferred embodiments, at least one of the sockets 20 is in fluid communication with microscale fluid flow channel 16, allowing for an operative component 21 to be in fluid communication with microscale fluid flow channel 16. In other embodiments, sockets 20 are in communication with each other, allowing for communication between operative components 12 in different sockets 20 and microscale fluid flow channel 16. The communication between sockets 20 may be fluidic, electrical, optical, acoustical or any other suitable method of communication that will become readily apparent to one skilled in the art, given the benefit of this disclosure.

In accordance with another preferred embodiment, substrate body 12 further includes at least one data port 4, and at least one data channel 6 within substrate body 12 in communication with data port 4 and at least one socket 20. Data port 4 may be in electrical or optical communication with data channel 6 and one or more socket 20. Data channel 6 and data port 4 may be bi-directional for both receiving and broadcasting data. Examples of such electrical communication include standard interfaces such as IR, PCMCIA, USB, serial, parallel, RS232, Firewire, etc.

It will be understood by those skilled in the art that the microfluidic substrate assemblies disclosed here may comprise numerous different sizes and geometries. For example, the substrate assemblies may be about 3½ inches by about 8½ inches, 3½ inches by 9½ inches, 3¼ inches by 4¾ inches, ⅝ inches by 1 inch, 4 inches by 6 inches, or may be a cartridge having the dimensions of a postage stamp, a PCMCIA card, or a credit card. The different size cartridges have innumerable uses and may be used in any of numerous devices. For example, in embodiments that are 3½ inches by 9½ inches, the cartridge may be suitable for use as a pumping manifold, e.g. pump heads, degasser, or flow meters; as injector manifolds, e.g. injector valves, pressure sensors, or detector flow cells; and as a pre-concentration manifold, e.g. flow-switching valves and pre-concentrators. In embodiments that are 3¼ inches by 4¾ inches, the substrate assemblies may be useful as a screening manifold, e.g. reagent and sample flow switching valves, mixers, reactors and the like. In embodiments that are about the size of a PCMCIA card, the substrate assemblies may be useful as capillary electrophoresis (CE) cartridges, e.g. CE columns; as conductivity cells; as sensors; as valves; as pre-concentration cartridges, e.g. valves, pre-concentration units, sensors, etc.; as dynamic field gradient focusing (DFGF) cartridges, e.g. DFGF units, valves, sensors; and the like.

Certain preferred embodiments are useful as sensors chips, e.g. pH, pO₂, pCO₂, dissolved pO2, dissolved pCO₂, salinity, conductivity, nitrate and phosphate sensors; as mixer chips, e.g. active ultrasonic mixers; and may perform any unit operations required by a separation system or other analytical device. Additionally, the substrate assemblies may be stainless steel for high pressure applications, may have rigid side walls or integral ridges to prevent polymer creep, may fit into a bed of a robotic handler, e.g. a robotic fluid handler, may be plug and play, and may have numerous fluid and electrical connectors as discussed here.

Referring to FIG. 2, in accordance with another preferred embodiment, substrate body 12 is generally planar, with sockets 20 arranged in a grid array located on an upper surface of planar substrate body 12. As used here, the term “generally planar” means card or cartridge-like, optionally being curvo-planar or otherwise irregular, but typically being rectilinear or right-cylindrical, and having a thickness less than about one third, preferably less than one quarter, more preferably less than about one fifth, e.g., about one sixth or less, the largest dimension of the major (i.e., largest) surface of the substrate. Directional references used here are for convenience only and not intended to limit the orientation in which the substrates are used. In general, the substrates can be used in any orientation; solely for purposes of discussion here, they are assumed to be in the orientation shown in the drawings appended hereto. The substrate body 12 may also further include an outlet port (not shown) in fluid communication with microscale fluid flow channel 16.

In the embodiment illustrated in FIG. 2, one or more operative components 21 are received in corresponding sockets 20. A housing 30 encapsulates microfluidic substrate assembly 10. Housing 30 serves to serves to both contain microfluidic substrate assembly 10 and protect it from high pressure or harsh environments. In other applications, housing 30 may serve to secure operative components 21 within sockets 20. In certain embodiments housing 30 is sealed shut. In other embodiments, potting compound is used to secure and protect the substrate body 12 within the housing 30.

In the illustrated embodiment, housing 30 is formed of first and second portions, which, when joined together, encapsulate microfluidic substrate assembly 10. The housing 30 is preferably made of a material capable of withstanding high pressure and harsh conditions. Examples of suitable materials include metals such as steel, e.g., stainless, galvanized, or other alloys. Other materials, such as plastics, e.g., PEEK, can also be used.

The addition of an operative component 21, optionally along with other such components in other sockets, allows microfluidic substrate assembly 10 to be easily configured for any of numerous applications. Having pre-formed sockets configured to receive operative components reduces the cost of adapting the substrate assembly for specific applications as well as allowing for further modification as needed. Through sockets 20, at least one or more of the operative components 21 are put in communication with microscale fluid flow channel 16. In certain embodiments, each operative component 21 is in communication with other operative components 21 that may be mounted in other sockets 20. The communication between operative components may be fluidic, electrical, optical, or any other form of communication. Other suitable forms of communication between operative components will be become readily apparent to those skilled in the art, given the benefit of this disclosure.

In accordance with certain preferred embodiments, it may beneficial for operative components 21 to be permanently mounted in sockets 20. This could prevent accidental or undesired third party modification. In some applications, microfluidic substrate assembly 10 may be subjected to extreme vibration, pressure or stress that could cause dismounting or misalignment of a non-permanently mounted operative component 21.

In other applications, it might be beneficial for an operative component 21 to be removable, allowing for field configuration of microfluidic substrate assembly 10 for a particular purpose, replenishment or replacement of spent components, or reuse of microfluidic substrate assembly 10 for another purpose. There are numerous ways of mounting and connecting operative components 21 to microfluidic substrate assembly 10 so as to provide communication with microscale fluid flow channel 16 and other sockets 20. In one embodiment, operative component 21 is securely mounted using a potting compound 19. The potting compound provides protection for operative component 21 as well as securing it. In certain other embodiments, the potting compound allows for re-entry.

Operative component 21 may be any number of devices depending on the particular application for which microfluidic substrate assembly 10 is being configured. In some applications, operative component 21 may be active upon fluid passing through microfluidic substrate assembly 10. Operative component 21 may test, analyze, filter, or otherwise treat the fluid. In other applications, operative component 21 may function to store, process or communicate data. There may also be multiple operative components 21 in microfluidic substrate assembly 10 performing any number of the above-mentioned activities in any combination.

In accordance with certain preferred embodiments, an operative component 21 associated with substrate body 12 is operative to pass fluid to or to receive fluid from a microscale fluid flow channel 16 of the substrate body, and can be a fluid reservoir. Such embodiments have application, for example, as highly advantageous microfluidic substrate assemblies for LC or other liquid separation devices, wherein operative component 21 can serve as a reservoir for eluting solvents, buffers, reagents, etc. It will be understood from this disclosure, however, that communication between microscale fluid flow channel 16 and an operative component 21 mounted on substrate body 12 need not necessarily be fluid communication, nor involve the flow of sample fluid between them, nor the discharge or injection of any liquid or other fluid from one to the other. Certain embodiments of the devices and methods disclosed here comprise reservoir type operative embodiments holding a solid reagent which can be dissolved during use, e.g., a replaceable solid reagent. Certain embodiments of the devices and methods disclosed here comprise reservoir type operative embodiments holding an enzyme or other catalyst.

Operative components in accordance with certain embodiments can include devices for generating fluid pressure in a microchannel of the substrate body, such as the high pressure observed in high performance liquid chromatography (HPLC) systems or the like. Suitable devices will depend, in part, on the specific use intended for the microfluidic substrate assembly and include micro-embodiments of so-called wax motors, also known as thermal actuators, heat capacitance motors or wax valve actuators. Such operative components generate pressure by the physical expansion of paraffin wax or the like as it changes from solid to liquid when heated within an enclosure such as a cylinder. The expanding wax is converted into mechanical force, which causes translation of a piston slidably mounted within the cylinder, thus creating hydrostatic pressure. Such devices are known, although their use in microfluidic substrate assemblies as disclosed here has not heretofore been suggested or recognized. Exemplary such devices include those disclosed in U.S. Pat. No. 5,222,362, U.S. Pat. No. 5,263,323, U.S. Pat. No. 5,505,706, and U.S. Pat. No. 5,738,658, the entire disclosure of each of these patents being incorporated herein by reference for all purposes. Other operative components in accordance with certain embodiments of the assemblies disclosed here include devices operative as a thermal actuator or a thermoelectric module for heating and/or cooling.

Other operative components in accordance with certain embodiments of the assemblies disclosed here include devices operative as a valve, pressure regulator, flow regulator, external port or plug, filter, trap or absorbent. While any operative component of the assemblies disclosed here may be either removable or permanently attached to the assembly (i.e., not removable from the assembly without damage to the component or to the assembly), operative components such as filters, traps or absorbents may be advantageously replaceable in various embodiments or may be designed to permit easy replacement of a filter material or absorbent material.

Other operative components in accordance with Certain embodiments of the assemblies disclosed here include, for example, one or more devices operative:
as components to degass (remove dissolved or evolved gases and/or bubbles) from fluid being treated or handled by the microfluidic assembly,
as a component to excite (e.g., fluorescence), illuminate (absorption source) or irradiate (e.g., microwave reactions or heating) a fluid being treated or handled by the microfluidic assembly,
as a component that is a miniaturized mass spectrometer,
as a component that is a NMR or MRI spectroscopy detector (e.g, a flowcell and a microcoil in combination), and/or
as a chromatographic, electrophoretic, isotachophoretic, isoelectric focusing, field gradient focusing or other separation column or chamber used for focusing and/or elution of molecules, particles, cells, organelles, or other species or objects.

Fluid communication between the microscale fluid flow channel and such actuators or like operative components integrated with the substrate body allows the fluid in the microscale fluid flow channel to be acted upon directly and physically. Exemplary of such devices are impellent devices, for example, any of various micro-pumps, such as micromachined pumps, diaphragm pumps, syringe pumps, and volume occlusion pumps. Other suitable pumps include a piezoelectric-driven silicon micropump that is bubble and particle tolerant and capable of pumping liquids at 1 mL/min. flow rates and commercially available from numerous sources such as FhG-IFT (Munich, Germany). Other pumping devices which can be employed as operative components in various embodiments of the microfluidic substrate assemblies disclosed here include endosmotic induced flow devices, devices which pump by electrochemical evolution of gases, and other pumping devices well known to those skilled in the art.

Other exemplary operative component devices include sensors for detecting or measuring a property or characteristic of fluid in the microchannel, or of a fraction or component of the fluid. Such sensors include, e.g., spectrographic sensors, such as sensors that include a light emitter passing light through a substantially transparent window or section of the microchannel and a light detector arranged opposite the emitter to receive and in some cases measure light. Such sensors and detectors, e.g. flow-cell detectors, are known, although their use in microfluidic substrate assemblies as disclosed here has not heretofore been suggested or recognized. Other sensors may include, for example, silicon based miniaturized devices for electrochemiluminescent detection.

Also exemplary of other operative component devices are acoustic transducers and reflectors and the like. Here, again, such devices are known, but their use in microfluidic substrate assemblies as disclosed here has not heretofore been suggested or recognized. Acoustic components suitable for generating a standing wave ultrasonic field transverse to the direction of flow in a microchannel are disclosed, for example, in International Patent Application number PCT/GB99/02384, the entire disclosure of which is incorporated herein by reference for all purposes. Such devices can be operative in certain embodiments of the microfluidic substrate assemblies disclosed here, when needed, to concentrate particles in fluid or to trap particles against a flow of suspending fluid.

The above mentioned and other components, which are generally commercially available, provide the building blocks of integrated systems in accordance with the present disclosure, for performing simple or complex chemical analyses. Certain exemplary microfluidic substrate assemblies in accordance with this disclosure comprise a micropump or other operative component. Currently, commercially available micropump technology suitable for incorporation into an operative component for at least certain embodiments of the microfluidic substrate assemblies disclosed here encompasses devices fabricated from any of a range of materials including polymers, and using methods that are mass fabrication compatible. Such micropumps typically can deliver both liquids and gasses (including chemically aggressive fluids) at flow rates in the order of 1 mL/min or less, are bubble and particle tolerant and can self-prime. Similarly, operative components can incorporate features to perform any of a spectrum of liquid handling requirements. This library of devices includes but is not limited to mixers, filters, stream splitters, injectors, droplet ejectors, solid phase extractors, liquid/liquid exchangers, micro-reactors, micro-chambers, micro-valves and de-bubblers. For example, suitable operative devices functional as micro-nozzles can be fabricated in silicon for droplet formation and ejection.

In addition, certain operative devices for certain preferred embodiments of the microfluidic substrate assemblies disclosed here are functional as flow sensors, e.g., flow meters capable of nanoliter precision, pressure sensors or thermal or temperature sensors. Exemplary such sensors may comprise one or more thermocouples. Micro-detectors also are available as sensor-type components for the devices disclosed here. For LC applications, several operative devices have been described. Certain operative devices suitable for use as sensors in various embodiments of the microfluidic substrate assemblies disclosed here are operative for pH, e.g., as an ISFET pH sensor, O₂, conductivity, etc. Certain operative devices suitable for use as sensors in various embodiments of the microfluidic substrate assemblies disclosed here are operative as acoustic, voltage or current-sensing electrodes or sensors. Certain operative devices suitable for use as sensors in various embodiments of the microfluidic substrate assemblies disclosed here are operative as chemical sensors, e.g., as nitrate, phosphate, or chloride sensors, etc. Certain preferred operative devices for the microfluidic substrate assemblies disclosed here are operative to perform electrochemical detection based on conductimetric, voltametric, redox, electrochemiluminescent, atomic emission and/or calorimetry detection principles. Other well-known detection methods known to those skilled in the art may also be incorporated into operative devices. In addition, miniaturized sensors with active sensing areas of a few microns can also be envisioned as detectors for LC applications. Numerous other sensors, including sensor type devices and the like, will be readily apparent to those skilled in the art given the benefit of this disclosure. It should be understood that any and all such sensors can be used in combination with each other in the microfluidic substrate assemblies disclosed here, just as it is true, more generally, that any and all of the operative components disclosed here can be used with each other in any combination or permutation suited to the intended application of the particular microfluidic substrate assembly.

In still other embodiments, an operative component is functional as an electronic memory component. In certain applications for example, it may be advantageous to record data in an operative component in one of the sockets of the substrate assembly, e.g., data about the substrate assembly, such as configuration, date of use, etc. In other applications, it may be advantageous to store data produced by the tests or activities performed by the substrate assembly. As used here, a memory component incorporated in an operative component is any device that is operative to store, read, write, and/or read and write information. Preferred memory units incorporated in an operative component include, but are not limited to, memory chips, e.g., read only memory (ROMs), programmable read only memory (ROMs) erasable programmable read-only memory (EPROMs), electrically erasable programmable read-only memory (EEPROMs), DIMMs, SIMMs, and other memory units and memory chips well known to those skilled in the art and commercially available from numerous manufacturers such as Siemens, Toshiba, Texas Instruments and Micron. Other suitable memory components and techniques for the use of encryption in the acquisition, storage and transmittal of data by or to the memory component may be found in the commonly assigned U.S. patents incorporated herein by reference. In other exemplary applications, the electronic memory component is specific for a specific HPLC system embodied in the microfluidic substrate device.

In other embodiments, an operative component of the microfludic substrate assembly is a microprocessor. In certain applications it may be advantageous to be able to perform computations on the data produced by the microfluidic substrate assembly. Other applications may require microprocessor-controlled activation of various steps of processing or testing. This level of functionality can be achieved by any of numerous commercially available microprocessors.

In other embodiments, an operative component of the microfluidic substrate assembly is an electronic tracking device. In certain applications it may be necessary or advantageous to track individual microfluidic substrate assemblies. In applications involving multiple microfluidic substrate assemblies, being able to locate and identify individual microfluidic substrate assemblies could be critical. An example of such a tracking device is a radio transponder. Other suitable tracking devices will be readily apparent to those skilled in the art given the benefit of this disclosure.

In other embodiments, an operative component of the micro fluidic substrate assembly is a communication device. In some applications it may be advantageous for the microfluidic substrate assembly to receive and/or transmit data collected by or stored on the microfluidic substrate assembly. In other applications, being able to remotely access and control the microfluidic substrate assembly would allow easier implementation in remote or difficult to get to areas. Examples of such communication technologies include, IR, RF, Bluetooth, as well as analog and Digital cellular technology.

Other operative component devices suitable for mounting aboard a microfluidic substrate assembly will be apparent to those skilled in the art given the benefit of this disclosure, and will depend in most cases largely upon the application or use intended for the microfluidic substrate assembly.

As noted above, multiple operative components may be mounted on a single microfluidic substrate assembly. Any combination of the above mentioned devices may be used as operative components. Some embodiments may not require all the sockets of a microfluidic substrate assembly to have an operative component mounted within. In accordance with another embodiment, sockets 20 not receiving an operative component 12 receive a plug 31, seen in FIG. 2. The plug(s) optionally are functional to maintain connectivity with the microscale fluid flow channel 16 and other sockets 20, or to function as a termination unit to maintain a fluid tight seal.

As noted above with respect to FIG. 1, substrate body 12 optionally includes at least one data port 4, and at least one data channel 6 within substrate body 12 in communication with the data port 4 and at least one of the sockets 20. Optionally each operative component 21 is in communication with data channel 6. The communication may be one way or bi-directional. The communication may be electrical, optical, or any other suitable form of communication that will become readily apparent to those skilled in the art given the benefit of this disclosure.

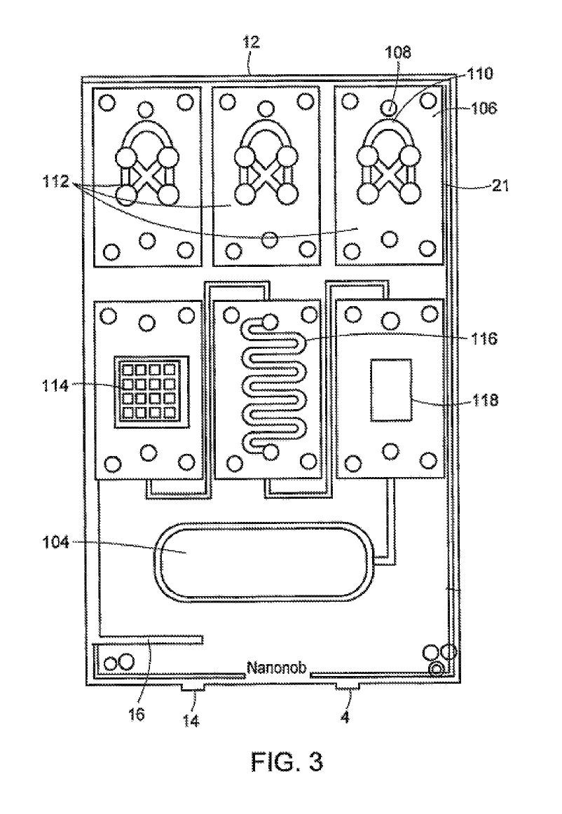

Referring to FIG. 3, an exemplary microfluidic substrate assembly 10 is shown, with multiple operative components 21 mounted on substrate body 12. The illustrated embodiment further includes a fluid reservoir 104 in substrate body 12. Each of the operative component includes a substrate body 106 defining at least one fluid input port 108, at least one microscale fluid flow channel 110 (these features of the operative components being labeled in FIG. 3 only for the operative component in upper right corner), and an operative device or feature. Shown in the embodiment of FIG. 3 as examples of operative devices are reagent stores 112, a sample preparation device 114, a separator 116, and a detector 118. As discussed above, the operative device can be any suitable device including, but not limited to, the exemplary devices listed here.

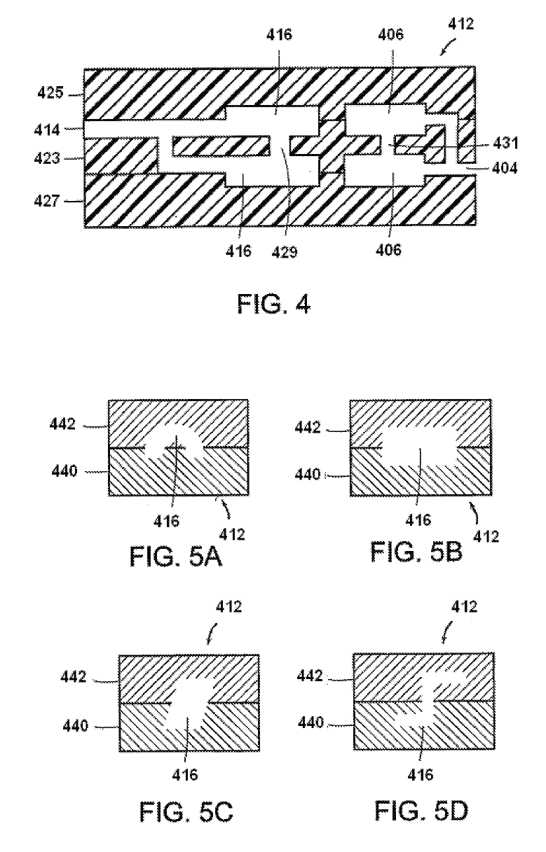

In accordance with another embodiment, as seen in FIG. 4, a microfluidic substrate assembly 10 includes at least one microscale fluid flow channel 16 at each of more than one level within the multi-layer substrate 12. In the illustrated embodiment, a central layer 23 is sandwiched between an upper layer 25 and a lower layer 27. A microscale fluid flow channel 16 is formed between upper layer 25 and central layer 23. A microscale fluid flow channel 16 is formed between central layer 23 and lower layer 27. At least one microscale fluid flow channel 16 at each of more than one level within multi-layer substrate 12 is in fluid communication with inlet port 14 for transport of fluid to be tested. At least one microchannel via 29 extends between levels within multi-layer substrate 12 for fluid communication between microscale fluid flow channels on different levels. A plurality of sockets (not shown) are provided, with each configured for receiving an operative component, wherein at least one socket is in communication with a microscale fluid flow channel 16.

In certain embodiments, substrate body 12 may further include at least one data channel 6 at each of more than one level within the multi-layered laminated substrate in communication with data port 4 and at least one socket 20. At least one data tap 31 extends between levels within substrate body 12 for communication between data channels 6 on different levels.

Microscale fluid flow channels and data channels are referred to in some instances as interlayer channels. In preferred embodiments, as illustrated in FIG. 4, the microscale fluid flow channels and data channels at each of multiple levels within the substrate are formed at the surface-to-surface interfaces between layers of the substrate. In the illustrated embodiment, two microchannels 16 and two data channels 6 are formed in central layer 23, formed, e.g., of PEEK or other plastic, having micromachined or micromilled grooves on both an upper and lower surface and sandwiched between two upper layer 25 and lower layer 27 of the substrate 12. Through-holes, micromachined or otherwise formed, in central layer 23 and passing from an upper surface groove to a lower surface groove provide fluid communication via 29 and data tap 31, respectively. In certain preferred embodiments one or both of the sandwiching layers 25, 27 of the substrate is a flexible sheet or film.

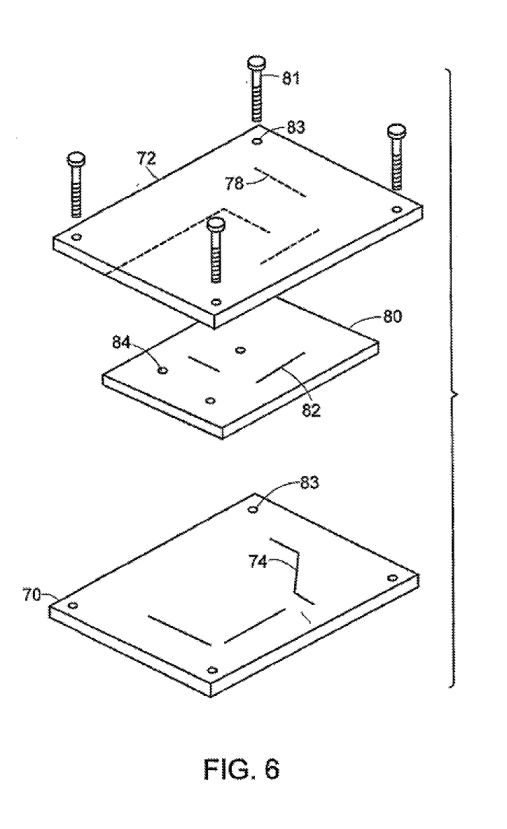

These interlayer microchannels 16 and data channels 6 may have any number of configurations such as straight, curvo-linear, serpentine or spiral depending on application. Their cross-sectional configuration may be regular or regular. Exemplary cross-sections of microchannels 16 and data channels 6 formed between a first layer 40 and a second layer 42 are seen in FIG. 5A-D, including semicircular, rectangular, rhomboid, and serpentine, respectively.

In accordance with certain preferred embodiments, in which at least one layer of multi-layered laminated substrate 12 is formed of plastic, micro fluidic substrate assembly 10 is operative and fluid tight at fluid pressure in microscale fluid flow channel 16 in excess of 100 psi. In other preferred embodiments, the multi-layer laminate of the d substrate is operative and fluid tight at pressure in microscale fluid flow channel 16 in excess of 200 psi, more preferably in excess of 300 psi, most preferably at a pressure greater than 500 psi. Certain preferred embodiments, including certain embodiments adapted to perform or for use in conjunction with chromatography and especially those embodiments wherein the multi-layered laminated substrate is sandwiched between steel plates, are operative and fluid tight even at pressures within the microscale fluid flow channels of the laminated substrate up to 2500 to 3000 psi, or even up to 5000 psi. As used here psi preferably refers to psi gauge as opposed to psi absolute. Especially preferred embodiments are operative, including being fluid-tight along the periphery of the microchannels within the substrate, even at fluid pressure in the microscale fluid flow channel in excess of 1000 psi.

In other preferred embodiments, as seen in FIG. 6, a multi-layer laminated substrate 80 is sandwiched between a pair of rigid plates 70, 72. Multi-layer laminated substrate 80 may include multiple layers of plastic welded one to another, with rigid plates 70, 72 sandwiching multi-layer laminated substrate 80 between them. Multi-layer laminated substrate 80 may be formed of layers of PEEK or other plastic, e.g., to form a 0.003-0.005 inch thick layer of PEEK. In still another embodiment, the multiple plastic layers of multi-layer laminated substrate 80 are selectively welded one to another to form a fluid-tight seal along a microchannel within the substrate. Employing plastic substrate layers in high-pressure embodiments provide significant advantages in manufacturing cost and flexibility

Optionally, the plastic layers of multi-layer laminated substrate 80 are welded (e.g., solvent welded, etc.) one to another, and rigid plates 70, 72 are formed of metal and are fastened directly to each other by fasteners, such as bolts 81 extending through apertures 83 formed in rigid plates 70, 72. As used here, direct fastening means that a bolt, latch or other fastener has compressive contact with the rigid sandwiching plates. Preferably, multiple bolts or the like extend from one to the other of the rigid sandwiching plates. In accordance with certain preferred embodiments, grooves for fluid flow channels can be micromachined, laser cut or otherwise milled or formed in the inside surface of one or both metal (or other rigid material) clamping plates that may be, e.g., 3/16 of an inch to 3 inches thick. In the illustrated embodiment, microgrooves 74, 82 and 78 are machined into the surfaces of rigid plate 70, multi-layer laminated substrate 80, and rigid plate 72, respectively. The cooperation of microgrooves 74, 82, 78 define fluid-tight microchannels of the resulting multi-layer laminated substrate assembly. Through holes or vias 84 in multi-layer laminated substrate 80 provide fluid communication from microchannels 78 on the lower or inside surface of rigid plate 72 to microchannels 74 on the upper or inside surface of rigid plate 70.

As noted above, in accordance with certain preferred embodiments, at least one layer of the multi-layer laminated substrate is formed of PEEK. PEEK is a high temperature resistant thermoplastic, which has superior chemical resistance allowing for its use in harsh chemical environments, and which retains its flexural and tensile properties at very high temperatures. PEEK is especially advantageous because it has a low glass transition temperature (Tg) and will weld at a temperature that will not lead to the distortion, warping, or destruction of environmentally sensitive elements contained within the plastic pieces. Glass, carbon fibers, carbon black, carbon particles, gold, titanium dioxide, etc., may be added to PEEK to enhance its mechanical and thermal properties. One advantage of using PEEK in the assembly of a fluid-handling substrate is that a selective JR welding process may be visually monitored, as PEEK in its amorphous form can be a sufficiently clear and optionally colorless material, allowing for visual inspection of the seals created by the welding process. Therefore, fluid-tight seals within the multi-layer substrate, such as those created using selective IR welding discussed elsewhere herein or other suitable methods, for example, may be inspected prior to further assembly of the fluid-handling substrate. In accordance with certain preferred embodiments, crystalline PEEK is employed as a. layer of the laminated substrate or a coating on another layer. Advantageously, crystalline PEEK provides good chemical resistance.

In certain embodiments, at least one PEEK layer includes an IR absorbing species in concentration sufficient for IR welding of the PEEK layer. The IR absorbing species may be distributed substantially homogeneously throughout the PEEK layer or disposed on the surface of the PEEK layer. Suitable IR absorbing species include, for example, dyes, zinc oxide, silicon oxide and metal species. A coating layer comprising the IR absorbing species may be distributed in a binder disposed on the surface of the PEEK layer. Examples of a coating layer are a spray coat, a stamping or a spin coat. In some embodiments the binder is formed of PEEK. In certain embodiments the IR absorbing species is deposited onto the surface of the PEEK layer by physical or chemical vapor deposition.

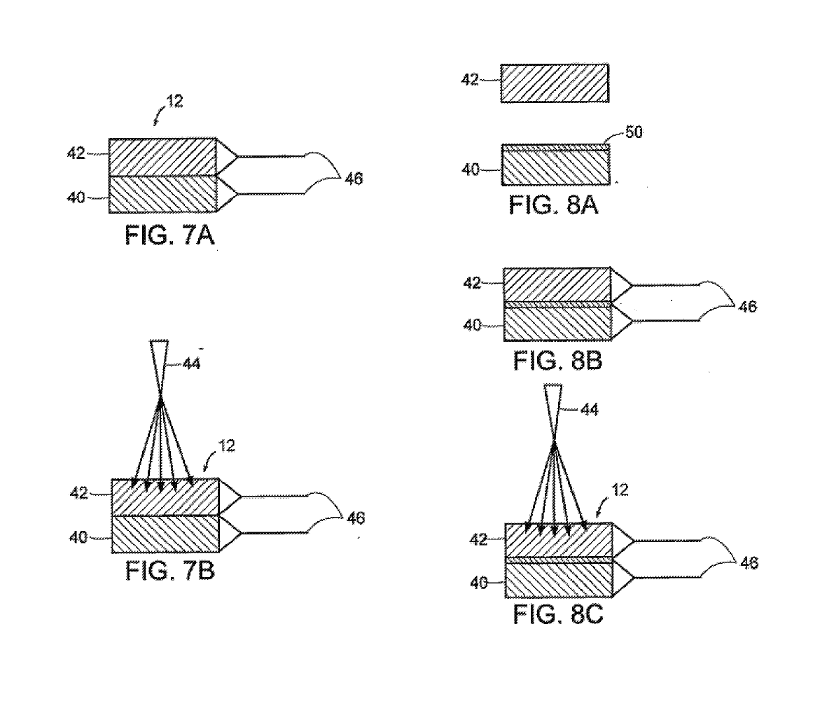

In accordance with another embodiment illustrated in FIGS. 7A-B, at least first and second layers 42, 40 of multi-layer laminated substrate 12 are selectively welded to each other to form a fluid-tight seal at least along one microchannel within multi-layer laminated substrate 12 (microchannel and other internal structures/components omitted for clarity). A suitable method for forming multi-layer laminated substrate 12 includes the steps of forming a surface-to-surface interface by aligning a surface of first substrate layer 42 against a surface of second substrate layer 40 using a mechanical device, such as an alignment stage 46, as seen in FIG. 7A. Second substrate layer 40 is capable of absorbing incident radiation, whereas first substrate layer 42 is energy transmissive. An electromagnetic (EM) beam 44 is applied through the surface of first substrate layer 42, and absorbed by second substrate layer 40, resulting in welding of the two layers to form a substrate sub-assembly having an internal fluid channel at the interface. Optionally, the sub-assembly is only partially exposed to radiation to heat only one or more selected portions of the interface to a temperature sufficient to weld the substrate components together, to form a fluid-tight seal between the substrate layers 40, 42 at the interface along the fluid microchannel.

In another embodiment shown in FIGS. 8A-C, the substrate may be formed by coating at least a selected area of the surface of first layer 40 with a radiation (EM) absorptive material coating 50 prior to forming the surface-to-surface interface. In this embodiment, both substrate layers 40, 42 are EM transmissive. Application of EM beam 44 heats the EM absorptive material coating 50, thereby welding the substrate layers 40, 42 together. In certain embodiments, absorptive material coating 50 is coated onto only one or more selected portions of the surface of the first substrate layer 40 and the sub-assembly is exposed non-selectively to JR radiation. Alternately, the absorptive material coating 50 is coated onto the entire surface of the first substrate layer 40 and only one or more selected portions of the interface are exposed to IR radiation. One method of achieving this involves exposing the sub-assembly to radiation through a mask having a configuration corresponding to the one or more selected portions of the interface. In certain preferred embodiments, the radiation absorptive material is IR-absorptive material and the radiation is IR radiation.

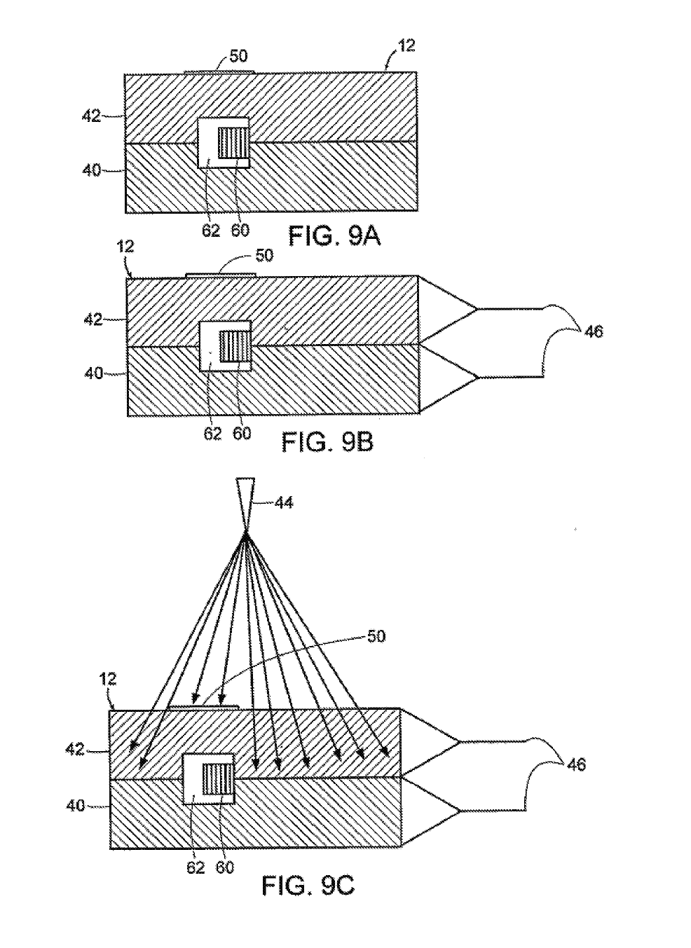

In accordance with certain preferred embodiments, as seen in FIGS. 9A-C, optionally contained within microchannel 62 is an environmentally sensitive element 60. As used herein, the term “environmentally sensitive element” refers to an element that would be destroyed if it were subjected to temperatures normally required to seal the plastic layers, and/or exposed to one or more fluids, e.g. strong acids, that might damage the element. Thus, for example, environmentally sensitive element 60 may be intolerant to the Tg of plastic layers made of PEEK. What is considered environmentally sensitive depends on the substrate material being welded, the temperatures and or pressure used during the welding, and on the species in a fluid that is introduced into the fluid handling substrate. Environmentally sensitive elements, as used here include, but are not limited to, the architecture features of the channels, fluids, soft gaskets, polyelectrolyte and other gels with valving sub-systems, flexible membranes, sensors with tiered membrane assemblages, electrical sensors, mechanical devices, biological components with sensor membranes, reagents for biotransformations, arrays of gene probes and analogues, detectors, and chromatography reagents. Certain sensors, whether electrical or biological, are also sensitive to high temperature and tend to be destroyed by the high temperatures. Fluids can also be sensitive to chemical adhesives and high temperatures of the current welding methods, and the composition of any adhesives added to effect welding of the pieces together may be altered by the incident radiation, for example, the adhesive may photoreact with the other components within plastic pieces. Some fluids are susceptible to chemical reactions under high temperature and pressure, and the resulting products could change the character and reactivity of the fluid. For example, chromatography reagents, such as beads with bonded phases, can be destroyed by high temperatures.

A portion of first layer 42 may be masked with absorptive material coating 50, and first and second layers 42, 40 may be aligned with alignment stage 46, as seen in FIG. 9B. The unmasked portions are exposed to EM beam 44 as seen in FIG. 9C and, therefore, only those locations are heated to seal the first and second layers 42, 40.

The layer(s) of the multi-laminated substrate in any of the above disclosed microfluidic substrate assemblies can be formed of numerous materials. Suitable materials include, for example, polysulphone, PEEK, PFE, polycarbonate, Teflon, stainless steel, PDMS, Pyrex, soda glass, CVD diamond, PZT, silicon nitride, silicon dioxide, silicon, polysilicon, Au, Ag, Pt, ITO, and Al. Any one or all of the layers can be made from such materials.

In accordance with another embodiment, the substrate body is molded out of desirable materials with the microchannel(s) and sockets defined by a temporary casting material. Once the substrate is formed and hardened, the temporary casting material can be removed using a method that does not affect the material of which the substrate body is formed. Temporary casting material can be any of a number of materials that can be chemically dissolved or melted using processes that do not affect the substrate body material. Pressure washing can remove any remaining residue. After the temporary casting material is dissolved and cleared, all that remains is the substrate with the now defined microchannel(s) and sockets. Methods using PEEK to form the substrate body may include using chemical solvents to which PEEK is impervious. Other methods utilize low temperature plastics that can be burned or melted at a temperature that does not affect PEEK. In still other embodiments, a spacer of Teflon® or other similar non-stick material can be used to define the microchannel(s). The advantage of using a material such as Teflon® is that the substrate material will not bond to it. Therefore, when the substrate body has been cast and allowed to set up, the Teflon® spacer can be removed from the substrate by simply extracting the Teflon® spacer.

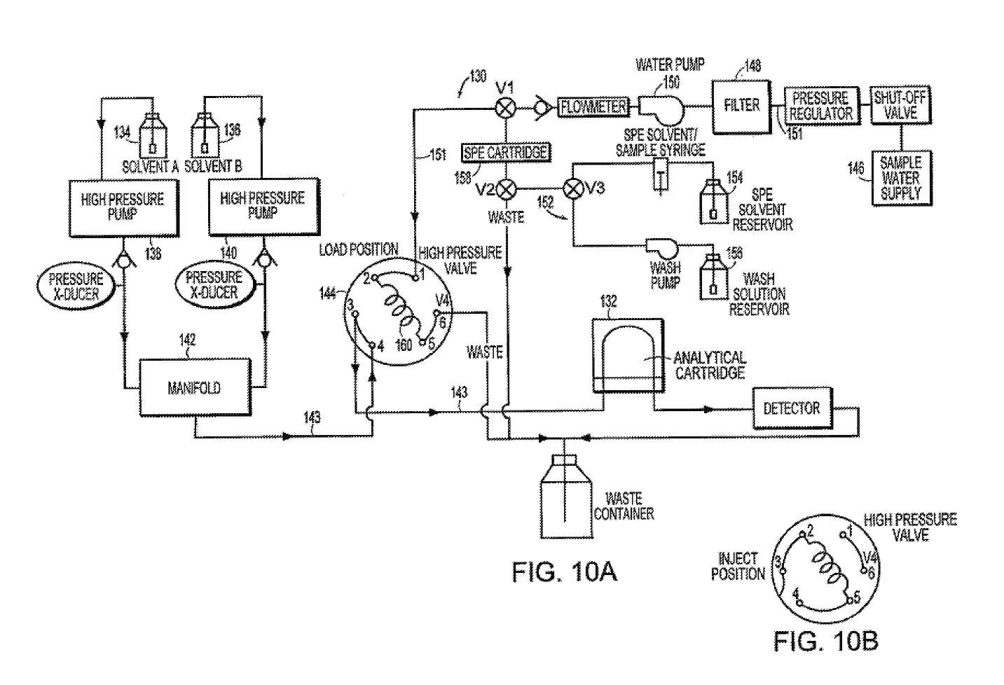

As seen in FIGS. 10A-B, an HPLC system 130 incorporates a microfluidic substrate assembly 132, referred to in FIG. 10A as an analytical cartridge. HPLC system 130 includes solvent supplies 134, 136 which provide a constant flow under high pressure by way of pumps 138, 140 and manifold 142 and high pressure solvent supply line 143 to high pressure valve 144. A sample water supply 146 in combination with a filter 148 and a pump 150 provides a sample via supply line 151 to high pressure valve 144. In certain preferred embodiments, a solid phase extraction (“SPE”) system 152 is incorporated in order to concentrate material found in the supply flow stream. SPE system 152 includes SPE solvent reservoir 154 and wash solution reservoir 156 which are passed through SPE cartridge 158 into supply line 151.

In the position illustrated in FIG. 10A, high pressure valve 144 is in a load position, in which coil 160 is filled with sample water, and solvent supply line 143 is bypassed by high pressure valve 144 directly to microfluidic substrate assembly 132. When a sample is desired, high pressure valve 144 is switched into the inject position, for a short interval, and coil 160 is connected in line with solvent supply line 143 such that a water sample is injected into supply line 143 and passed on to microfluidic substrate assembly 132 for analysis and/or testing. High pressure valve 144 is switched back into the load position after the sample has been introduced into supply line 143.

An example of a fluid-handling substrate assembly, in the form of a fluid separation microfluidic substrate assembly, interfaced with an analytical system, e.g. a chromatography system, is shown in FIG. 11. Such an analytical system typically is positioned within an end-user's facility for automated analysis. That is, the analytical system may be positioned near, or in-line with, the sample flow itself, such that analysis of samples may occur automatically, e.g. using auto-samplers, auto-injectors, and the like, or to facilitate rapid analysis of samples, e.g. sampling during a process by an operator at an end-user's facility. The system can be configured for analysis at specified intervals, e.g. every minute, hour, day, etc., such that continuous monitoring of a process can be performed with little or no user input. That is, the system can be configured to run a test such as a chromatographic method at a specified time interval without additional input from an operator.

Referring to FIG. 11, an analytical system 200 typically includes a multi-layer laminated microfluidic substrate assembly 210, interfaced with an analytical device, e.g. a chromatography instrument. Numerous mechanisms for interfacing microfluidic substrate assembly 210 with analytical system 200 are suitable, and exemplary interfaces are described below. Microfluidic substrate assembly 210 may be designed using the methods described above, for example, by etching microchannels into two or more layers of PEEK and assembling the layers, using selective IR welding for example, to form a microfluidic flow channel at the interface of the layers. Subsequently, a packing material may be introduced into microfluidic substrate assembly 210 to form a separation cartridge operative to separate species in a fluid.

Analytical system 200 optionally includes a treatment unit 202, such as a filter, a guard column, a solid phase extraction silo for analyte pre-concentration, etc. Treatment unit 202 may contain a plurality of single use solid phase extraction cartridges, corresponding to solid phase extraction cartridge 158 described above with respect to FIG. 10B. Analytes may be pre-concentrated such that trace levels of analyte are concentrated to levels that are detectable by analytical system 200. That is, the concentration of an analyte may be increased b 10¹, 10², 10³ 10⁴, 10⁵, 10⁶, 10⁷, 10⁸, 10⁹ times or higher in order to reach levels that are easily detected using a detector of analytical system 200. The treatment units are optional and may be replaced with other chromatographic devices, such as, for example, guard columns, filters, semi-permeable membranes, etc. Alternatively, the treatment units can be replaced with a fluid flow channel such that little or no operations are performed on the fluid prior to entry into microfluidic substrate assembly 210.

Analytical system 200 also typically includes a graphical user interface 204 for programming the system and/or monitoring system performance. The graphical interface may take numerous forms such as, for example, a keypad, an LCD screen, a touch screen, etc. In certain embodiments, the graphical user interface is omitted and the information on microfluidic substrate assembly 210 is used to program analytical system 200. Analytical system 200 optionally contains a receiver/transmitter 206 to provide for remote operation and diagnosis, e.g., operation of analytical system 200 over the Internet and/or transmission of data over the Internet to a remote facility. In certain embodiments, microfluidic substrate assembly 210 itself is a receiver/transmitter, and thus the receiver/transmitter of analytical system 200 may be omitted.

Analytical system 200 typically includes at least one detector 208. The type of detector used typically depends on the optical and physical properties of the species in the fluid. Additionally, the detectors are usually interchangeable such that the detector may be switched to a different type of detector, e.g. from a UV-Visible absorbance detector to a fluorescence detector. Suitable detectors include but are not limited to UV-Visible absorbance detectors, IR detectors, fluorescence detectors, electrochemical detectors, voltammetric detectors, coulometric detectors, potentiometric detectors, thermal detectors, ionization detectors, NMR detectors, EPR detectors, Raman detectors, refractive index detectors, ultrasonic detectors, photothermal detectors, photoacoustic detectors, evaporative light scattering detectors, mass-spectrometric detectors, and the like. Microfluidic substrate assembly 210 typically interfaces with analytical system 200 through a manifold 256, which is discussed in detail below with respect to FIG. 12. In alternative embodiments, however, microfluidic substrate assembly 210 can interface directly with analytical system 200, i.e., it can be connected directly to a fluid supply source, e.g. a pump and/or injector, without any intervening mechanical components.

A closeable face plate 215 may be hingeably or removably attached to analytical system 200 and can be closed over, or around, analytical system 200 to protect it from harsh environmental conditions, such as chemical solvents, UV radiation and the like. A power and communication interface 216 supplies power and data to analytical system 200. Such interfaces typically are operative to provide a power source to analytical system 200, and can also provide communication between analytical system 200 and a central computer, e.g. a computer in communication with analytical system 200 for monitoring test results and/or for exchanging information with analytical system 200.

To achieve high reproducibility, a fixed-loop injector 214 is typically used to introduce sample into analytical system 200. Suitable fixed-loop injectors are well known to those skilled in the art and are commercially available from numerous sources, e.g. Beckman Instruments (Fullerton, Calif.). Other injectors may be used in place of the fixed-loop injector depending on the intended use of analytical system 200. For example, auto-injectors and/or auto-samplers may be used to provide for automated sampling and analysis of fluids. Suitable auto-samplers and auto-injectors are well known to those skilled in the art and are commercially available from numerous manufacturers. Optionally, analytical system 200 can be programmed such that the auto-samplers and/or auto-injectors take samples at specified intervals, e.g. every 10 seconds, every minute, hourly, daily, weekly, monthly, etc., such that testing of the fluid can be performed without any input from a user. Analytical system 200 also includes precise microfluidics for accurate solvent gradients and includes solvent reservoirs and/or reagent magazines 218 for providing a fluid phase for running the chromatographic methods of microfluidic substrate assembly 210, e.g. solvent gradients and the like. Such precise microfluidics can be achieved using numerous methods known to those skilled in the art, such as the methods described in the commonly assigned U.S. patents incorporated herein by reference for all purposes. As discussed above, one or more pumps are typically in fluid communication with the solvent reservoirs, and are operative to generate a fluid flow.

Typically the installation of analytical system 200 can be customized such that analytical system 200 can be positioned in numerous places in a facility. That is, the dimensions and shapes of analytical system 200 can be designed for placement in numerous areas of an operating facility, and the functions, e.g. chromatographic methods, of analytical system 200 can be tailored to perform innumerable tests desired by an end-user. In preferred embodiments, analytical system 200 is placed near the sample or process to be monitored. That is, analytical system 200 may be placed, either fixably or removably mounted, for example, near the fluid to be analyzed. For example, analytical system 200 can be custom mounted to a conduit 220 that carries a fluid sample, e.g. river water, out of a manufacturing facility, for example. Depending upon its configuration, analytical system 200 can automatically sample the fluid flowing through the conduit, e.g. using an auto-sampler, auto-injector and the like, or one or more valves positioned in the conduit can be connected to analytical system 200 for introducing samples. Alternatively, an operator can manually take samples from the conduit and can introduce the samples through a fixed-loop injector, for example, using a needle, syringe, and the like. One skilled in the art given the benefit of this disclosure will be able to select suitable positions for analytical system 200 described here depending on the type of analyses to be performed.

A microfluidic substrate assembly typically interfaces with an analytical system through a manifold. As seen in FIG. 12, microfluidic substrate assembly 252 interfaces with a. multi-layer laminated manifold 256. Referring to HPLC system 130 in FIG. 10A, such a manifold would be the interface between microfluidic substrate assembly 132 and high-pressure supply line 143. Multi-layer manifold 256 may be assembled using any of the methods described above and other methods known to those skilled in the art. Thus, FIG. 12 shows a first multi-layer laminated assembly, e.g., microfluidic substrate assembly 252, interfaced to a second multi-layer laminated assembly, the manifold 256. As discussed, manifold 256 is seen in the particular embodiment of FIG. 12 to be a multi-layer laminated structure and has one or more microfluidic channels (not shown) for introducing fluid into or receiving fluid from microfluidic substrate assembly 252.

Manifold 256 may comprise a first layer 258 attached to a second layer 259, which itself is attached to a third layer 260. As can be seen in FIG. 12, the second layer 259 typically is sandwiched between the first layer 258 and the third layer 260. Fluid channels (not shown) can be provided within and/or at the interface(s) of the layers of such manifolds as described above. For example, layer 259 in manifold 256 can optionally be constructed as. a microfluidic substrate assembly as described above, optionally with layer 259 being formed substantially of PEEK. The layers of the multi-layer laminated manifold each can be manufactured from any of numerous materials, including but not limited to PEEK, steel, e.g. stainless steel, and the like. Different layers of multi-layer laminated manifold 256 may be formed of different materials.

In certain embodiments, microfluidic flow channel extends between two or more of the layers, e.g., a microfluidic flow channel can extend from the third layer into the second layer and optionally into the first layer, for example. A microfluidic flow channel can be formed in one or more of the layers using numerous techniques, e.g. UV embossing, micro-machining, micro-milling, and the like. For example, a microfluidic flow channel can be etched into the second layer and the first layer such that when the second layer is assembled to the first layer a fluid-tight microfluidic flow channel is created. As discussed above, the layers can be assembled to form the multi-layer laminated manifold. For example, the layers can be assembled by welding the layers together, optionally with a gasket positioned between the layers, or can be assembled using adhesives and the like. One skilled in the art given the benefit of this disclosure will be able to select suitable methods for assembling the layers of multi-layer laminated manifolds suitable for use with the multi-layer microfluidic substrate assemblies disclosed here.

Preferably, the manifold includes at least a first microfluidic channel in fluid communication with a solvent reservoir and with an input orifice of the microfluidic substrate assembly. Thus, solvent may flow into the microfluidic substrate assembly through a microfluidic channel in the manifold, e.g. by pumping the fluid into the microfluidic substrate assembly using a pump. The manifold can include a second microfluidic channel that is in fluid communication with an output orifice of the microfluidic substrate assembly and typically is also in fluid communication with a detector. Therefore, a sample may be introduced into the microfluidic substrate assembly through the first microfluidic channel in the multi-layer manifold, separated by the microfluidic substrate assembly, and the separated species can flow out of the microfluidic substrate assembly through the second microfluidic channel in the manifold to a detector that can measure the amount and nature of the species present in the sample. Thus, as discussed above, the fluid handling substrates described here may be configured to interface with an analytical system in numerous ways, e.g. through a manifold 256 or a microfluidic substrate assembly 252 or both. One skilled in the art given the benefit of this disclosure will be able to design other suitable manifolds and devices for interfacing the microfluidic substrate assembly with an analytical system.

In certain embodiments, an interface 254 is mounted to manifold 256. Interface 254 typically is operative to create a fluid-tight seal when microfluidic substrate assembly 252 is plugged into manifold 256. That is, interface 254 is operative to provide a sealing force suitable to prevent fluid from leaking between manifold 256 and microfluidic substrate assembly 252. Optionally, one or more gaskets can be positioned between microfluidic substrate assembly 252 and interface 254 to aid in forming a fluid-tight seal. Interface 254 may also be formed as a multi-layer laminated structure. Thus, in certain embodiments, a plurality of multi-layer laminated structures may be in fluid communication with each other, through microchannels, ports, and the like, and with one or more analytical systems. One skilled in the art, given the benefit of this disclosure, will be able to select suitable mechanisms for retaining microfluidic substrate assembly 252 against manifold 256 and/or interface 254 of manifold 256 to create a fluid-tight seal. Exemplary mechanisms include cams, springs, pressure plates, welding, clamps, and combinations of any of them.

As discussed above, in alternative embodiments microfluidic substrate assembly 252 is plugged directly into the system without using a manifold. For example, suitable connectors may be added to microfluidic substrate assembly 252 such that it can be in direct fluid communication with a flow line, e.g. a flow line including one or more solvents and one or more species to be separated. One skilled in the art, given the benefit of this disclosure, will be able to select suitable mechanisms and devices for interfacing microfluidic substrate assembly 252 to an analytical system.

In other embodiments, the manifold itself is in communication with a second component-on-board; such as a device that is operative to generate or control fluid flow. For example, as seen in FIG. 13, a pump or valve actuator 270 can be attached to multi-layer laminated manifold 256 and can be configured such that fluid is drawn or passed from a fluid reservoir, e.g. a solvent reservoir, and forced or passed into manifold 256 and subsequently into microfluidic substrate assembly 252. In addition to pumps and valve actuators, such devices may be any of the devices known to those skilled in the art and discussed above, including but not limited to vacuum manifolds and the like. The device for generating or controlling fluid flow can also be in communication with one or more injectors as discussed above.

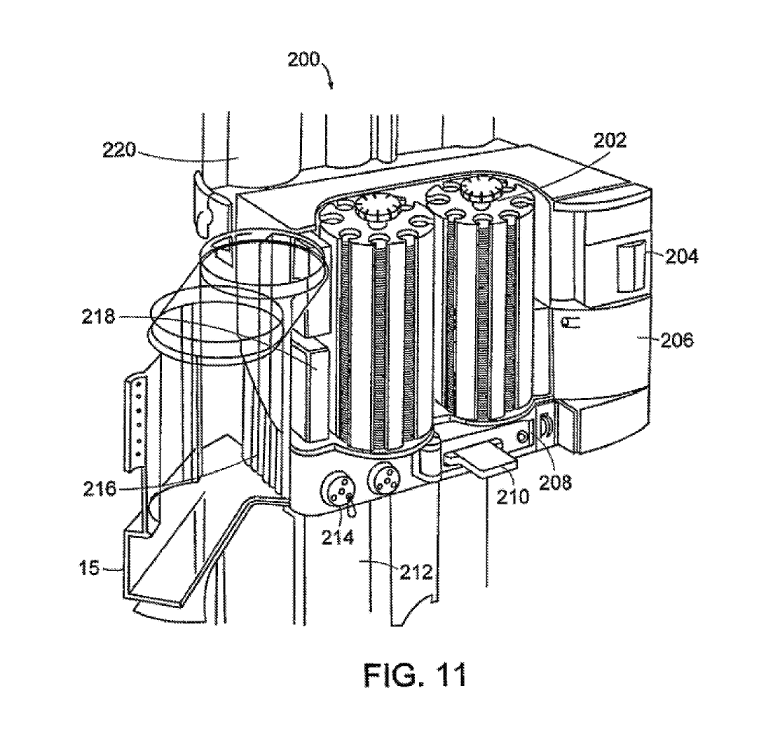

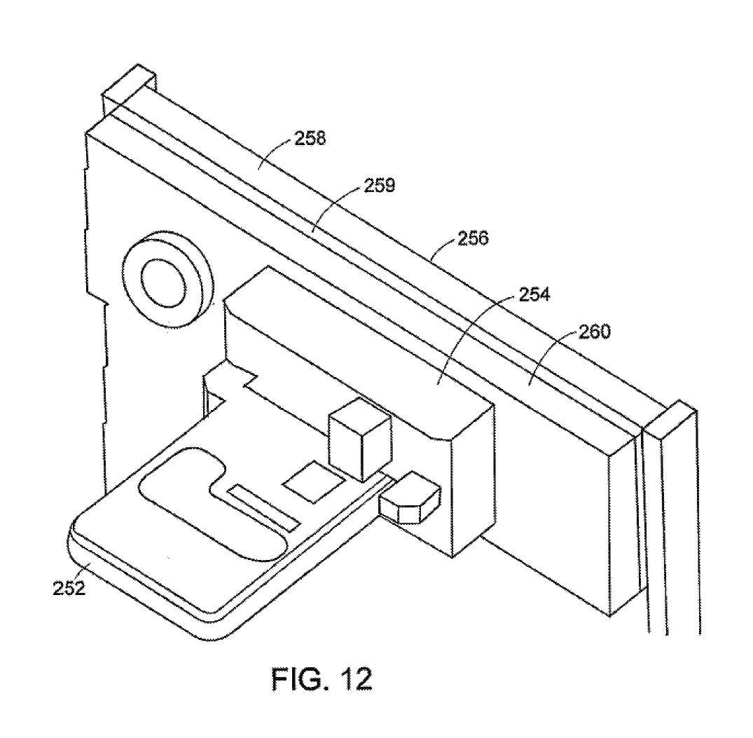

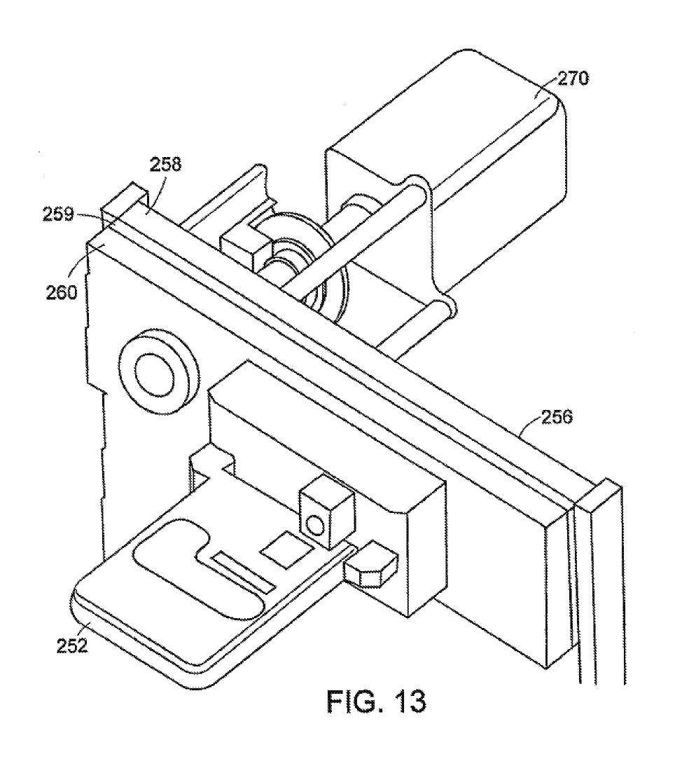

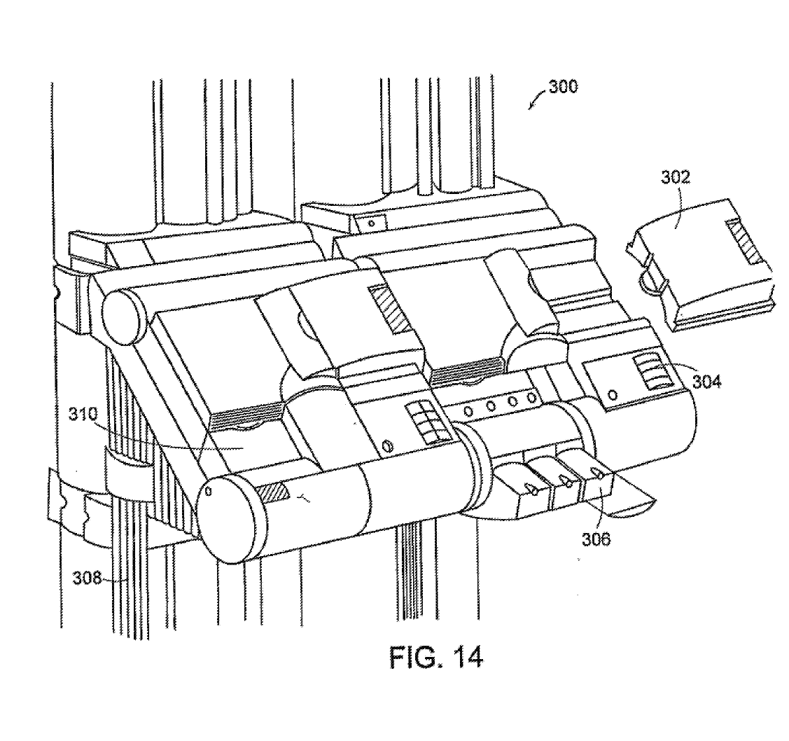

An additional example of a microfluidic substrate assembly, assembled in accordance with this disclosure, interfaced with an analytical system is shown in FIG. 14. An analytical system 300 includes a microfluidic substrate assembly 302, shown here encapsulated in a housing as a cartridge. Microfluidic substrate assembly 302 may be, e.g., an assembly operative to perform capillary electrophoresis or capillary liquid chromatography or capillary liquid electrochromatography.

Analytical systems in accordance with this disclosure may optionally include a graphical user interface 304 and buffer cassettes 306. Graphical user interface 304 can be used to program the system and/or microfluidic substrate assembly 302 for a specific method, e.g. a specific voltage program or solvent gradient, run time, flow rate, and the like. As discussed above, graphical user interface 304 can be omitted in embodiments where microfluidic substrate assembly 302 is operative to program the system, e.g., where microfluidic substrate assembly 302 includes an analytical method in a memory unit. Buffer cassettes 306 are equivalent to solvent reservoirs. That is, buffer cassettes 306 may be loaded with any suitable mobile phase needed to perform an electrochromatographic method, for example. Preferably, the mobile phases are different in different buffer cassettes such that solvent gradients or other variations can be implemented in the analytical method. Buffer cassettes 306 may be in communication with one or more devices that are operative to generate a fluid flow (not shown), e.g. pumps and the like.

Analytical system 300 typically has one or more power and communication interfaces 308 and can be custom installed at a user's facility such that automated analyses may take place or such that the system is positioned near the fluid to be analyzed. As discussed above, communication interface 308 may send and/or receive data to or from a central computer, or other device. Analytical system 300 can be controlled by remote operation and diagnosis using a communication device 310 by various methods, such as for example, e-mail over the Internet. Communication device 310 typically is used to alter the method of analytical system 300 without having to manually enter the new method using the graphical user interface. This feature provides for remote configuration, or reconfiguration as the case may be, of analytical system 300. In certain embodiments, communication device 310 is omitted and analytical system 300 is controlled by information sent from microfluidic substrate assembly 302, which may include its own communication device positioned with a chamber in microfluidic substrate assembly 302, to analytical system 300.

The size of microfluidic substrate assembly 302 can be tailored such that it has the appropriate dimensions, e.g. height, width and thickness, and has the appropriate connectors to interface with any analytical system. For example, in embodiments comprising a capillary column, the dimensions of microfluidic substrate assembly 302 may be reduced such that its footprint is smaller and occupies less space on analytical system 300. Suitable fluid connectors including those discussed here, e.g. male/female connectors and the like, can be attached to microfluidic substrate assembly 302 and are typically operative to create a fluid-tight seal between microfluidic substrate assembly 302 and analytical system 300. Suitable electrical connectors can be attached to microfluidic substrate assembly 302 including those discussed above, for example, PCMCIA connectors, USB connectors, serial connectors and the like. The electrical connectors typically provide for transfer of information to and from microfluidic substrate assembly 302.

As discussed above, microfluidic substrate assembly 302 can interface with the system through a manifold, such as manifold 256 shown in FIG. 12, or can interface with the system directly, e.g. without any intervening physical components. Suitable connectors for interfacing with a manifold can be positioned on any surface of the housing, unit of microfluidic substrate assembly 302. Microfluidic substrate assembly 302 may include one or more connectors on a major surface, e.g., the back surface of microfluidic substrate assembly 302 shown in FIG. 14, such that microfluidic substrate assembly 302 can interface with a manifold and sit flush with the surface of analytical system 300. For example, microfluidic substrate assembly 302 may have outwardly projecting connectors that plug into a manifold, having a receiving socket, positioned on analytical system 300. When microfluidic substrate assembly 302 is plugged into the manifold, microfluidic substrate assembly 302 snaps into position on analytical system 300, e.g., becomes seated in a slot on the surface of analytical system 300. Thus, microfluidic substrate assembly 302 is in fluid communication with analytical system 300 and is retained by the system such that vibrations will not dislodge microfluidic substrate assembly 302 from the system, i.e., microfluidic substrate assembly 302 remains in fluid communication with analytical system 300 even in the presence of vibrations or other physical disturbances. Numerous other devices, e.g., cams, pulleys, springs, pressure plates and the like may be used to retain microfluidic substrate assembly 302 against the manifold of analytical system 300 such that a fluid tight seal is preserved.

Although the present invention has been described above in terms of specific embodiments, it is anticipated that other uses, alterations and modifications thereof will become apparent to those skilled in the art given the benefit of this disclosure. Such alterations are intended to include the interchanging of one or more of the components of any of the embodiments with the components of any of the other embodiments disclosed here. It is intended that the following claims be read as covering such alterations and modifications as fall within the true spirit and scope of the invention. It is intended that the articles “a” and “an”, as used below in the claims, cover both the singular and plural forms of the nouns which the articles modify.

1. A microfluidic substrate assembly comprising:
a substrate body having a surface and comprising:
at least one fluid inlet port;
at least one microscale fluid flow channel within the substrate in fluid communication with the inlet port; and
a plurality of sockets in the surface of the substrate body, each socket comprising a corresponding recess into the substrate body, and each socket configured to receive an operative component, wherein at least one of the plurality of sockets is in communication with the microscale fluid flow channel.

2. The microfluidic substrate assembly of claim 1, wherein the substrate body is a multi-layer laminated substrate.

3. The microfluidic substrate assembly of claim 1, further comprising a housing, the substrate body being positioned in the housing.

4. The microfluidic substrate assembly of claim 1, wherein the substrate assembly is generally planar.

5. (canceled)

6. The microfluidic substrate assembly of claim 5, wherein the plurality of sockets are located in a grid array.

7. The microfluidic substrate assembly of claim 1, wherein at least one of the sockets is in fluid communication with the microscale fluid flow channel.

8. The microfluidic substrate assembly of claim 1, wherein at least one of the sockets is in fluid communication with at least one other of the sockets.

9. The microfluidic substrate assembly of claim 8, wherein multiple sockets of the sockets have the same configuration.

10. The microfluidic substrate assembly of claim 1, wherein at least one of the sockets is in electrical communication with at least one other of the sockets.

11. The microfluidic substrate assembly of claim 8, wherein at least one of the sockets is in optical communication with at least one other of the sockets.

12. The microfluidic substrate assembly of claim 1, wherein the substrate body further includes at least one fluid outlet port in fluid communication with the fluid inlet port.

13. The microfluidic substrate assembly of claim 1, further comprising a fluid reservoir in fluid communication with the microscale fluid flow channel.

14. The microfluidic substrate assembly of claim 1, wherein the substrate body is formed of PEEK.

15. The microfluidic substrate assembly of claim 1, wherein the substrate body further comprises:
at least one data port; and
at least one data channel within the substrate body in communication with the data port and at least one of the sockets.

16. The microfluidic substrate assembly of claim 15, wherein the data channel is in electrical communication with the data port.

17. The microfluidic substrate assembly of claim 15, wherein the data channel is in optical communication with the data port.

18. The microfluidic substrate assembly of claim 15, wherein the data channel is in electrical communication with at least one of the sockets.

19. The microfluidic substrate assembly of claim 15, wherein the data channel is in optical communication with at least one of the sockets.

20. The microfluidic substrate assembly of claim 15, wherein the data channel is bi-directional.

21. The microfluidic substrate assembly of claim 15, wherein the substrate body further comprises a data output port in communication with the data channel.

22. A microfluidic substrate assembly comprising:
a generally planar multi-layer laminated substrate having a surface comprising:
at least one fluid inlet port;
at least one microscale fluid flow channel at each of multiple levels within the multi-layer substrate, in fluid communication with the inlet port for transport of fluid to be tested;
at least one microscale via extending between levels within the multi-layer laminated substrate for fluid communication between microscale fluid flow channels on different levels; and
a plurality of sockets in the surface of the substrate body, each socket comprising a corresponding recess into the substrate body, and each socket configured to receive an operative component, wherein at least one of the plurality of sockets is in communication with at least one microscale fluid flow channel.

23. The microfluidic substrate assembly of claim 22, wherein the multi-layer laminated substrate further comprises:
at least one data port; and
at least one data channel at each of more than one level within the multi-layered laminated substrate in communication with the data port and at least one of the sockets; and
at least one data tap extending between levels within the multi-layered laminated substrate for communication between data channels on different levels.

24. The microfluidic substrate assembly of claim 22, wherein at least one layer of the multi-layered laminated substrate is formed of plastic and the substrate assembly is operative and fluid tight at a fluid pressure in the microscale fluid flow channels in excess of 100 psig.

25. The microfluidic substrate assembly of claim 22, wherein each of the sockets is in communication with at least one other of the sockets.

26. The microfluidic substrate assembly of claim 22, wherein the microfluidic substrate assembly further comprises a pair of rigid plates, the laminated substrate being sandwiched between the rigid plates.

27. The microfluidic substrate assembly of claim 22, wherein at least one layer of the multi-layer laminated substrate is formed of PEEK.

28. The microfluidic substrate assembly of claim 27, wherein the at least one PEEK layer comprises an IR absorbing species in a concentration sufficient for IR welding of the PEEK layer.

29. The microfluidic substrate assembly of claim 28, wherein a coating layer comprising the IR absorbing species distributed in a binder is disposed on the surface of the PEEK layer.

30. The microfluidic substrate assembly of claim 22, wherein at least first and second layers of the multi-layer laminated substrate are selectively welded to each other to form a fluid-tight seal at least along one microscale fluid flow channel within the multi-layer laminated substrate.

31. A microfluidic substrate assembly comprising:
a substrate body with a surface comprising:
at least one fluid inlet port;
at least one microscale fluid flow channel within the substrate body in fluid communication with at least one fluid inlet port for transport of fluid to be tested;
a plurality of sockets in the surface of the substrate body, each socket comprising a corresponding recess into the substrate body, and each configured for receiving an operative component and in communication with at least another of the sockets, wherein at least one of the plurality of sockets is in communication with the microscale fluid flow channel; and
at least one operative component mounted in a corresponding one of the sockets.

32. The microfluidic substrate assembly of claim 31, wherein the substrate body comprises a multi-layer laminated substrate.

33. The microfluidic substrate assembly of claim 31, wherein the at least one of the sockets is in fluid communication with the microscale fluid flow channel.

34. The microfluidic substrate assembly of claim 31, wherein the at least one operative component comprises a fluid reservoir.

35. The microfluidic substrate assembly of claim 31, wherein the at least one operative component comprises a solid reagent suitable to be dissolved during use of the assembly.

36. The microfluidic substrate assembly of claim 31, wherein the at least one operative component holds an enzyme, catalyst or other reagent.

37. The microfluidic substrate assembly of claim 31, wherein the substrate body further comprises at least one fluid outlet port in fluid communication with at least one fluid inlet port.

38. The microfluidic substrate assembly of claim 31, wherein the at least one operative component is operative as a sensor.

39. The microfluidic substrate assembly of claim 31, wherein the at least one operative component is operative as a light sensor across a microscale fluid flow channel within the substrate body.

40. The microfluidic substrate assembly of claim 31, wherein the at least one operative component is operative as a flow sensor, pressure sensor, thermal sensor, temperature sensor, pH sensor, O₂ sensor, conductivity sensor, acoustic sensor, voltage sensor, current sensor, chemical sensor, or electrochemical sensor.

41. The microfluidic substrate assembly of claim 31, wherein the at least one operative component is operative as a sensor for detection based on at least conductimetric, voltametric, redox, electrochemiluminescent, atomic emission or calorimetry detection principles.

42. The microfluidic substrate assembly of claim 31, wherein the at least one operative component is operative as an ultrasonic actuator or transducer across a microscale fluid flow channel within the substrate body.

43. The microfluidic substrate assembly of claim 31, wherein the at least one operative component is operative to generate fluid pressure in a microchannel of the substrate body.

44. The microfluidic substrate assembly of claim 31, wherein the at least one operative component is operative as a valve, pressure regulator, flow regulator, external port or plug, filter, trap or absorbant.

45. The microfluidic substrate assembly of claim 31, wherein the at least one operative component comprises a thermal actuator or a thermoelectric module for heating or cooling.

46. The microfluidic substrate assembly of claim 31, wherein the at least one operative component comprises a device operative at least:
as a component to degas fluid being treated or handled by the microfluidic assembly,
as a component to excite, illuminate or irradiate a fluid being treated or handled by the microfluidic assembly,
as a component that is a miniaturized mass spectrometer,
as a component that is a NMR or MRI spectroscopy detector or
as a separation column or chamber.

47. The microfluidic substrate assembly of claim 31, wherein the at least one operative component is an impellent device.

48. The microfluidic substrate assembly of claim 31, wherein the at least one operative component is operative to directly contact a fluid in the microscale fluid flow channel.

49. The microfluidic substrate assembly of claim 31, wherein the at least one operative component is one of a micromachined pump, diaphragm pump, syringe pump and a volume occlusion pump.

50. The microfluidic substrate assembly of claim 31, wherein the at least one operative component is operative to induce flow in a microscale fluid flow channel by one of endosmotically and electrochemical evolution of gases.

51. The microfluidic substrate assembly of claim 31, wherein the operative component is permanently mounted in a socket.

52. The microfluidic substrate assembly of claim 51, wherein the operative component is permanently mounted in a socket using potting compound

53. The microfluidic substrate assembly of claim 31, wherein the operative component is removably mounted in a socket.

54. The microfluidic substrate assembly of claim 31, wherein the operative component is an electronic memory component.

55. The microfluidic substrate assembly of claim 54, wherein the electronic memory component is a read only memory component.

56. The microfluidic substrate assembly of claim 54, wherein the electronic memory component is a read/write memory component.

57. The microfluidic substrate assembly of claim 31, wherein the operative component is a microprocessor.

58. The microfluidic substrate assembly of claim 31, wherein the operative component is an electronic tracking device.

59. The microfluidic substrate assembly of claim 31, wherein each of the sockets not receiving an operative component receives a plug.

60. The microfluidic substrate assembly of claim 31, wherein the substrate body further comprises:
at least one data port; and
at least one data channel within the substrate body in communication with at least one data port and at least one of the sockets.

61. The microfluidic substrate assembly of claim 31, wherein the operative component comprises:
a substrate body defining:
at least one fluid input port;
at least one microscale fluid flow channel; and
at least one operative device.

62. A microfluidic substrate assembly comprising:
a substrate body having a surface and comprising:
at least one fluid inlet port;
at least one microscale fluid flow channel within the substrate in fluid communication with the inlet port; and
a plurality of sockets in the surface of the substrate body, each socket comprising a corresponding recess into the substrate body, and each socket configured to receive an operative component, wherein at least one socket is in fluid communication with the microscale fluid flow channel;
a focusing operative component received by at least one of the sockets, operative to focus molecules, particles, cells, organelles, or other species from fluid received from the microscale fluid flow channel; and
a detecting operative component comprising a flow cell with an NMR microcoil, the detecting operative component received by a second one of the plurality of sockets, the second one of the plurality of sockets being in fluid communication with the socket receiving the focusing operative component, the detecting operative component being operative to detect or measure properties or characteristics of species received from the focusing operative component.

63. The microfluidic substrate assembly of claim 46, wherein the device is operative at least as a component that is a NMR or MRI spectroscopy detector and comprises a flowcell and a microcoil in combination.

64. The microfluidic substrate assembly of claim 46, wherein the device is operative at least as a chromatographic, electrophoretic, isotachophoretic, isoelectric focusing, field gradient focusing or other separation column or chamber

65. The microfluidic substrate assembly of claim 64, wherein the device is operative at least for focusing or elution of molecules, particles, cells, organelles, or other species.
